(19) United States
(12) Patent Application Publication
(10) Pub. No.: US 2014/0302482 A1
(43) Pub. Date: Oct. 9, 2014

(54) ELECTROMAGNETIC BLOOD PRESERVATION AND STORAGE

(71) Applicants: Dan Dietz, Richmond, TX (US); George Daniel O'Clock, Waconia, MN (US)

(72) Inventors: Dan Dietz, Richmond, TX (US); George Daniel O'Clock, Waconia, MN (US)

(21) Appl. No.: 13/988,992

(22) PCT Filed: Nov. 23, 2011
(86) PCT No.: PCT/US11/62141

§ 371 Date: Apr. 9, 2014

Related U.S. Application Data
(63) Continuation of application No. 12/952,382, filed on Nov. 23, 2010.
(60) Provisional application No. 61/263,450, filed on Nov. 23, 2009.
(60) Provisional application No. 61/409,838, filed on Nov. 3, 2010.

(51) Int. Cl. C12N 13/00

(52) U.S. Cl. CPC C12N 13/00
USPC 435/2; 435/307.1

(57) ABSTRACT

Embodiments of the present invention include method, system and apparatus for storing fluids (including whole blood or any of the components thereof) to thereby enhance viability of the fluids being stored outside a living body. Embodiments of the present invention include, for example, exposing an electric, magnetic, electromagnetic, field or combinations thereof to one or more storage mediums containing the fluids. Embodiments of the fields exposed to the blood or components thereof thereby causing one or more cells to be repelled from the field to reduce the risk of the one or more cells from sticking together. Embodiments of the claimed invention advantageously extend the viability or shelf life of the blood or components thereof. Embodiments of the present invention can further include a pump for circulating the fluids and a warmer to maintain a constant temperature.

### RELATED APPLICATIONS

This application is a continuation in part of and claims priority to U.S. patent application Ser. No. 12/952,382, titled “Electromagnetic Blood Preservation and Storage” filed on Nov. 23, 2010, which claims benefit and priority to U.S. Provisional Patent Application No. U.S. 61/263,450, titled the same and filed Nov. 23, 2009, and U.S. Provisional Patent Application No. 61/409,838, titled the same and filed Nov. 3, 2010, and is related to U.S. patent application Ser. No. 12/433,566, titled “Devices and Methods for Treating Magnetic Poising and/or Magnetically Inducing Rouleaux” filed Apr. 30, 2009, each and all of which are incorporated by reference in their entireties.

### TECHNICAL FIELD

The present disclosure relates generally to blood preservation and storage.

### BACKGROUND

Whole blood and blood components from a donor are commonly preserved and stored under refrigeration until they are required by a patient receiving the transfusion. Blood storage under refrigeration generally depletes the metabolites used by the blood during circulation in the body to maintain red blood cell (RBC) viability and function, and at the same time generates waste products that would otherwise be removed in the body. Sterile solutions containing anticoagulant and/or preservative systems are generally used in an attempt to maintain RBC viability and decrease the possibility of bacterial contamination.

Alterations in RBC biochemistry and physical properties that occur during storage are generally referred to as “storage lesions.” Refrigeration slows but does not stop RBC metabolism, and RBCs in storage continue to metabolize glucose through the anaerobic glycolysis pathway, producing two adenosine diphosphates (ADPs, from adenosine triphosphate or ATP) and a lactic acid during the metabolism of each glucose to 2,3-biphosphoglycerate (BPG, also referred to as DPG) or 1-phosphoglycerate (PG). BPG, which is widely accepted as necessary for allostearic facilitation of oxygen release from RBC in the body, is favored by a higher pH, whereas a lower pH favors PG, which has no effect on the oxygen dissociation curve. The, tendency for pH in stored RBC to drop over time is only partially inhibited by the buffering capacity of the preservative and additive solutions currently in use.

ATP levels also decline during RBC storage, depleting at the expiration date to only from 45 to 86 percent of the original levels, depending on the storage additives used. The expiration date of RBC storage is typically three to six weeks from the blood being withdrawn from the donor. While low ATP levels are associated with poor RBC viability, a high ATP level does not necessarily indicate good viability because of other types of storage lesions. Sodium and potassium leak through the membranes of the RBCs, elevating the potassium levels in the storage solution. BPG levels, generally associated with pH, may stay almost normal during the first week of storage, but also decline to the expiration date. Decreased BPG levels are associated with a left-shift in the oxygen dissociation curve of hemoglobin, resulting in an inhibited ability to release oxygen in the tissues of the recipient until circulation restores normal BPG levels, which can take up to 24 hours after transfusion.

Also, plasma hemoglobin levels continually increase due to RBC hemolysis that continues during storage. Blood ammonia levels also increase during storage. Further, RBCs manifest physical changes during storage, including the appearance of RBCs called echinocytes, which have multiple spiny projections; or the appearance of spherocytes, which take on a spherical shape as opposed to the normal biconcave disc shape of a healthy RBC.

RBCs in the body generally last about 120 days before hemolysis. However, the shelf life of RBCs in the available storage protocols is at most 3 to 6 weeks. Furthermore, recent studies have suggested that morbidity and mortality statistically increase with the length of storage of the RBCs, i.e., their storage age, prior to transfusion, especially after 1-2 weeks in storage.

Oxyhemoglobin (oxyHb) prevalent in arterial blood is diamagnetic with a reported susceptibility of −((0.13 to 0.65)×10-8 cgs emu/cm3 Oe; whereas deoxyhemoglobin (deoxyHb) which occurs predominantly in venous blood, following oxygen release in the capillaries, is paramagnetic with a reported susceptibility of +(13 to 33)×10-8 cgs emu/cm3 Oe. Methemoglobin, (metHb) in which the heme is essentially irreversibly oxidized, is also paramagnetic with susceptibility similar to that of oxyHb. The effects of strong magnetic fields, e.g., 30 to 100 kG, on blood have been reported in literature as including orientation of red blood cells and platelets with the magnetic field direction, polymerization and alignment of fibrinogens, and increasing the apparent viscosity of blood. For example, in an article titled “Effects of a static magnetic field of either polarity on skin microcirculation,” by Mayrovitz et al., reported in the Microvascular Research, vol. 69, pp. 24-27 (2005), reported a reduction in skin blood perfusion upon exposure of the patient to a neodymium magnet with a surface field of more than 4 kG.

There remains a long-felt and dire need in the art to inhibit the degradation of stored blood and blood components, to lengthen the shelf life, to improve the viability of RBCs in storage, to reduce the occurrence of complications associated with transfusions, and/or to reduce morbidity and mortality outcomes in transfusion recipients.

### SUMMARY

Embodiments of the present invention include, for example, methods, system, and apparatuses for improving the viability and/or shelf life of stored red blood cells (RBC or RBCs) by electromagnetically treating the blood in storage. For example, embodiments of the present invention exposes the blood or any components thereof continuous or periodic to electric, magnetic, or electromagnetic fields. Embodiments of the present invention also include, for example, an apparatus for storing blood comprising an electromagnetic generator to continuously or periodically generate electromagnetic stimulation in a blood storage compartment and/or blood flow path, e.g., an electrical current, magnetic field or combination thereof.

The Applicant has determined that the deterioration of RBC in storage, i.e., the period of time following collection from a donor until transfusion into a recipient patient, may arise at least in part from an extended period of electromagnetic inactivity or quiescence, which is termed “electromagnetic senescence” herein. This phenomenon might be explained as a gradual degaussing or loss of surface polarization of the RBC, or a loss of magnetization of the heme centers in the hemoglobins, and/or a redistribution of polarity, although embodiments of the present invention are not to be limited by this particular theory. The RBC in venous blood collected for blood banking and eventual transfusion, containing some deoxyhemoglobin, has an external surface orientation or polarity that helps keep the blood cells from sticking together due to the mutual repulsion of the like surface polarity. As the blood travels through the circulatory system of a living being, it is constantly cycled through bioelectromagnetic processing parameters that keep the heme irons magnetized and reconditioned for readily holding and releasing oxygen in repeated cycles through the cardiovascular circulatory system.

In the tissue or organ capillaries outside the lungs of a living being, such as during conventional blood storage protocol, the RBC are forced in close proximity to the internal surfaces of the capillary, oxygen is released and carbon dioxide taken on. The cells forming the capillary comprise a single-cell layer, and have bioelectromagnetic activity with intracellular electrical potential reported to be as much as 3 million ev/m in human cells. The capillary cells, and possibly to a lesser extent the surrounding tissue cells, are thus capable of bioelectromagnetic stimulation of the magnetically susceptible RBC, in addition to the electrical current incidental to the cardiac cycle and other neural and/or muscular activity. This is consistent with the observation that oxygen is more readily released in the vicinity of active muscles and/or organs where it is needed most.

Applicant further recognizes, for example, that the bioelectromagnetic stimulation may induce the hemoglobin in the magnetically susceptible RBC to roll or turn so that the external polarity is switched from negative or north (diamagnetic) to positive or south (paramagnetic) to facilitate release of the oxygen in the tissues. In the vicinity of the lungs and heart, the cardiac cycle can be a source of the bioelectromagnetic stimulation of the magnetically susceptible RBC, as well as the capillary cells, which are thought to stimulate the magnetization of the heme irons to facilitate carbon dioxide release and oxygen absorption. Once the blood is withdrawn from a vein and collected, however, the RBC in conventional collection and storage systems and methodologies are no longer subjected to the repetitive bioelectromagnetic stimulation experienced in normal circulation through the body of a living being. Thus, the magnetic and electrical properties of the RBC in storage can be gradually altered, and the hemoglobin observed to rapidly deteriorate and lose the ability to selectively bind and release oxygen and carbon dioxide.

Accordingly, embodiments of the present invention are directed to, for example, system, methods, and apparatus, of enhancing the storage fluids by exposing the fluids to bioelectromganetic stimulation having properties similar to those properties of the circulatory system of a living being. As used herein, the term “fluid” means whole blood or any of the components or substances contained therein or combinations thereof, including, but not limited to, plasma, erythrocytes, leukocytes, thrombocytes, proteins, carbohydrates (such as glucose or dextrose), mineral ions, hormones, carbon dioxide, platelets, albumin, blood-clotting factors, immunoglobulins, lipoprotein particles, hemoglobin, oxygen, nitric oxide, bicarbonate ions, and electrolytes.

According to an embodiment of the present invention, RBC in stored blood or blood components is continuously or periodically subjected to electromagnetic field, electric, and/or magnetic s, similar in magnitude and phase characteristics to those experienced in the body to constantly rejuvenate the RBC and maintain heme iron magnetization and/or external-internal polarity. In an embodiment, the applied electromagnetic stimulation serves to maintain the heme iron magnetization and/or surface magnetic polarity of the RBC, inhibiting electromagnetic senescence, preserving the RBC and inhibiting deterioration of the RBC as understood by those skilled in the art.

### BRIEF DESCRIPTION OF THE DRAWINGS

So that the manner in which the features and benefits of the invention, as well as others which will become apparent, may be understood in more detail, a more particular description of the invention briefly summarized above may be had by reference to the embodiments thereof which are illustrated in the appended drawings, which form a part of this specification. It is also to be noted, however, that the drawings illustrate only various embodiments of the invention and are therefore not to be considered limiting of the invention's scope as it may include other effective embodiments as well.

FIG. 1 schematically illustrates a blood storage and electromagnetic charge stimulation of blood cells according to an embodiment of the invention.

FIG. 2 is a side view of the blood storage compartment shown in FIG. 1.

FIG. 3 schematically shows a side section of tubing blood storage compartment including a source of electromagnetic field, electric, and/or magnetic for magnetically stimulating the blood during storage according to an embodiment.

FIG. 4 schematically shows a blood storage system with a blood circulation circuit for treating the blood during storage according to an embodiment.

FIG. 5 shows a red blood cell passing through a capillary tube with an externally opposing magnetic surface field according to an embodiment.

FIG. 6 shows a red blood cell passing through a capillary tube with an externally opposing magnetic surface field according to an alternate embodiment.

### DETAILED DESCRIPTION

Embodiments of the present invention include, for example, methods, apparatus, and systems of fluids by electromagnetically stimulating the RBC of the fluids to improve viability.

Embodiments of the present invention include, for example, periodically or continuously exposing fluids to an electromagnetic field, electric, and/or magnetic for an exposure time. As understood by those skilled in the art, electromagnetic field, has both an electric field component and a magnetic field component; where approximately half of the energy is in the electric field component and the other half of the energy is in the magnetic field component. As understood by those skilled in the art, in an electric field most of the field energy is in the electric field. As understood by those skilled in the art, in a magnetic field, most of the field energy is in the magnetic field. Embodiments, as understood by those skilled in the art, of the magnetic field can be, for example, in a range of about 0.5 to 500 Gauss; embodiments of the electromagnetic field can be, for example, in a range of about 15-100 Watts/m²; and the electric field can be in a range, for example, of about 3-300 volts/m.

As used herein, the term “exposure time” means exposure to an electromagnetic field, electric, and/or magnetic for a period of time of at least ten minutes, at least 30 minutes, at least 60 minutes, at least 2 hours, at least 5 hours, at least 10 hours, at least 20 hours, at least 24 hours, at least 48 hours, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 41 days, at least 42 days, at least 43 days, at least 44 days, at least 45 days, at least 46 days, at least 47 days, at least 48 days, at least 49 days, at least 50 days, at least 51 days, at least 52 days, at least 53 days, at least 54 days, at least 55 days, at least 56 days, at least 57 days, at least 58 days, at least 59 days, at least 60 days, at least 100 days.

Applicant recognizes, for example, that the lifecycle RBC is typically about 100 days. Outside the circulatory system of a living being, such as during conventional blood storage protocols, the lifecycle of RBC is much shorter. Accordingly, an embodiment of the present invention is directed to slow degeneration of one or more components of fluids during storage of the fluids outside the living being so that the degeneration of the one or more components more similarly emulates the degeneration of the one or more components by a circulatory system of a living being. As used herein, the tefin “slow degeneration” means a count of whole blood components with storage lesions (for example, but not limited to, autohemolysis or increased membrane permeability of the erythrocytes) for fluids stored in accordance to the methods, systems, and apparatus in accordance with embodiments of the present invention described herein that is lower than the count of storage lesions of fluids stored under the conventional storage methods (as described, for example, in the Background section). The slow degeneration, for example, to, can include but not be limited to at least 20%, 35%, or 40% more viable cells after, for example, 40 days of being stored when compared to the number of storage lesions stored for the same number of days using conventional storage methods.

Embodiments of the present invention can include, for example, an electric current that has amperage, voltage, wave form or a combination thereof corresponding to a cardiac cycle of RBC for a living being. In embodiments, the current is direct current or alternating current. In an embodiment, the current is pulsed at a frequency from 0.001 to 10 Hz. In an embodiment, the current is supplied at a voltage potential between from 1 to 1000 millivolts. An alternative embodiment, can include, for example, periodically or continuously applying a magnetic field to the RBC. As understood by those in the art, the magnetic field can be a static magnetic field or an oscillating magnetic field. As understood by those skilled in the art, the magnetic field according to embodiments of the claimed invention can also be a homogenous magnetic field or a heterogeneous magnetic field. Embodiments of the magnetic field can include, for example, a range of from about 0.5 to about 500 Gauss. In the alternative, embodiments of the magnetic field can include, for example, a range of from about 10 to about 100 Gauss. In an embodiment, the magnetic field is pulsed at a frequency from 0.001 to 10 Hz. In one embodiment, the electromagnetic stimulation is applied to the RBC just prior to or during transfusion into the recipient.

The storage of fluids in accordance with embodiments of the present invention can also include the presence of an added anticoagulant, pH buffer, nutrient, preservative, pathogen inactivator or combination thereof. In an embodiment, the RBC are stored in whole blood. The RBC can be stored, for example, in the presence of citrate-potassium-dextrose solution (CPD) such as CPD-1 or citrate-potassium-dextrose-adenine solution (CPDA) such as CPDA-1. In another embodiment, the RBC can be separated from whole blood, e.g. by centrifugation or aphoresis. In an alternative embodiment of the present invention, the RBC can be stored in the presence of adenine-saline solution (AS), e.g., AS-1, AS-2, AS-3, AS-4, AS-5, AS-6, as understood by those skilled in the art. In an embodiment, the method can include the step of inactivating pathogens, e.g., viruses, bacteria, parasites and so on, such as for example by adding a pathogen inactivator, such as in the Cerus INTERCEPT blood system, to the storage medium. Pathogen inactivators and inactivation methods are disclosed in U.S. Pat. No. 7,611,831, U.S. Pat. No. 7,293,985, U.S. Patent Pub. Application No. 2004/029897, U.S. Pub. Application No 2003/082510, U.S. Pub. Application No. 2003/113704, U.S. Pat. No. 6,951,713, U.S. Pat. No. 6,709,810, WO Patent Pub. Application No. 0191775, and U.S. Pat. No. 6,420,570, each and all of which are incorporated by reference in their entireties.

As understood by those skilled in the art, various processing techniques can be used to process RBC. For example, RBC can be process by contacting RBC with a rejuvenation solution, such as pyruvate-inosine-phosphate-adenine solution (PIPA), and irradiating the RBC. Storage methods in accordance with embodiments of the present invention can also include, for example, gas exchanging RBC to add or remove oxygen, carbon dioxide or a combination thereof. Embodiments of the present invention can also include, for example, dialysis to remove waste products from the RBC.

In an embodiment, the method may include agitating or pumping a medium such as plasma comprising the RBC and exposing the medium through an electromagnetic stimulation zone.

Embodiments of storing fluids can also including controlling the temperature of the fluids. For example, embodiments of the present invention can include maintaining the temperature of the fluids at about a range of about 96.5 to about 99.5 degrees Fahrenheit. Alternatively, embodiments of the present invention can include maintaining the temperature of the fluids at about a range of about 1 and 6° C., or between about 30 and about 40° C.

Embodiments of the present invention can be used to store fluids outside a living being a period of time in excess of 35 days. Embodiments of the present invention can be used to store fluids outside a living being a period of time in excess of 45 days and up to about 100 days. Embodiments of the present invention are effective such that the RBC and/or the storage media comprise more than 84% viable cells (as determined 24 hours post transfusion) following storage for 42 days, a pH greater than 6.98 following storage for 42 days, an ATP content greater than 86 percent of original ATP content at 21 days of storage, an ATP content greater than 60 percent of original ATP content at 42 days of storage, a 2,3-biphosp hog lycerate (BPG) content greater than 44 percent of original BPG content at 21 days of storage, a BPG content greater than 10 percent of original BPG content at 42 days of storage, a plasma potassium concentration less than 21 mmol/L at 21 days of storage, a plasma potassium concentration less than 45 mmol/L at 42 days of storage, a plasma hemoglobin concentration less than 191 ng/L at 21 days of storage, a plasma hemoglobin concentration less than 386 ng/L at 42 days of storage, or any combination thereof.

Embodiments of the claimed invention can further include an apparatus for storing fluids, the apparatus having the fluids contained. The apparatus can include, for example, one or more conductive wires so that an electromagnetic, electric, and/or magnetic field is produce and exposed to the fluids contained in the apparatus when an electric current is applied to the one or more conductive wires. The conductive wires, for example, can be positioned so that the electromagnetic, electric, and/or magnetic field generated therefrom is exposed evenly on a surface of the apparatus. As will be understood by those skilled in the art, the fluids can be exposed for an exposure time to improve viability and shelf life of the stored fluids.

In an embodiment, the RBC storage apparatus comprises an electric source to pass an electric current through the storage media, and the electric source can if desired include a controller to provide an amperage, voltage, wave faun or a combination thereof corresponding to a cardiac cycle for of RBC while in a living being. In embodiments, the electric source provides direct current or alternating current. The electric source in embodiments can include a controller to pulse the current at a frequency from 0.001 to 10 Hz; and/or to provide the current at a voltage potential between from 1 to 1000 millivolts.

In another embodiment, the RBC storage apparatus can additionally or alternatively comprise a signal generator to apply a magnetic, electric, and/or electromagnetic field to the fluids as understood by those skilled in the art. In embodiments, the magnetic field comprises a static magnetic field or an oscillating magnetic field. The magnetic field can be either a homogenous magnetic field or a heterogeneous magnetic field. In exemplary embodiments, the magnetic field is within a range of from about 0.5 to about 500 Gauss or within a range of from about 10 to about 100 Gauss. The magnetic field generator in one example comprises a controller to pulse the magnetic field at a frequency from about 0.001 to 10 Hz.

Embodiments of the present invention can include, for example, an electromagnetic stimulation zone to apply the electromagnetic stimulation to the RBC just prior to or during transfusion into the recipient.

As understood by those skilled in the art, a preservative solution such as, for example, citrate-potassium-dextrose solution (CPD) or citrate-potassium-dextrose-adenine solution (CPDA) or adenine-saline solution (AS) can be added. Further embodiments can include, for example, an anticoagulant, pH buffer, nutrient, preservative, pathogen inactivator or combination thereof.

In an additional or alternative embodiment can also include, for example, adding rejuvenation solution.

Embodiments of the present invention can further include, for example, a gas exchange zone to add to the RBC or remove from the RBC oxygen, carbon dioxide or a combination thereof. In another embodiment, the RBC storage apparatus can further comprise a dialysis zone to remove waste products from the RBC. The gas exchange zone and/or the dialysis zone can be components of a closed, fluid loop circuit, for example.

In an additional or alternative embodiment, the RBC storage apparatus can comprise a shear zone, as understood by those skilled in the art, to agitate the storage media comprising the RBC. System and method embodiments can include, for example, a pump to pump or circulate the storage media through an RBC flow circuit. Circulating the fluids to provide constant or periodic motion can, for example, reduce the risk of one or more cells of the fluids from settling and aggregation of the fluids, and also mitigates the problems of hot spots of the fluids.

In additional or alternative embodiments of the apparatus, a temperature control circuit is provided to maintain the temperature of the RBC, for example, between 1 and 6° C., or between about 30 and about 40° C.

In an embodiment, the electrical current or magnetic field applied to the fluid corresponds to the current or field applied to blood by the heart, either in a healthy heart or in the specific transfusion recipient, for example, a frequency and duration within 50% (i.e., 0.5 to 1.5 times the natural frequency or duration) or within 25% (i.e., 0.75 to 1.25 times the natural frequency or duration) of the electrical currents or fields ordinarily applied to blood from the atrioventricular node as it passes through the heart. In another embodiment, the strength of the current or field applied to the RBC is greater than that naturally applied in the right or left ventricle or right or left atrium, for example, 25, 50 or 100% greater, or from about twice to about 10 times greater, but not too great as to damage or injure the RBC, e.g. to avoid rouleaux. In an embodiment, the stored blood is periodically or continuously electrified or magnetized with a current and/or field effective to extend the life of the stored RBC. In other embodiments, the current or field is applied periodically to preserve the RBC, for example, from 1 to 5 seconds every 1 to 60 minutes or every 2 to 10 minutes, or from 30 seconds to 2 or 5 minutes every 1 to 12 hours, or for any duration and periodicity effective to improve the preservation and/or quality of the stored RBC. In an embodiment the electrical current and/or field are effective to inhibit charge depletion of the surface of the RBC, and in a further embodiment the electrical current and/or field are effective to maintain the magnetization levels of the heme irons in the RBC.

Thus, a patient can bank blood for autologous transfusion further in advance of surgery than is possible with conventional blood storage techniques, allowing the patient to fully recover from the blood loss. Further, the banked blood can be stored with a greater level of preservation or quality, which in one embodiment can be seen in the maintenance of uniform polarity of the RBC external surfaces. In other embodiments, the RBC have improved parameters indicative of viability, relative to conventional blood storage and preservation techniques, e.g., an increased proportion of viable cells (as determined 24 hours post transfusion) following storage, less pH loss or variation following storage, a greater ATP content greater relative to the original ATP content at collection, a greater 2,3-biphosphoglycerate (BPG) content relative to the original BPG content at storage, a lower plasma potassium concentration, a lower plasma hemoglobin concentration, or any combination thereof.

In an embodiment, the electromagnetic stimulation is applied to the blood as it is being transfused into the recipient, or just prior to transfusion, or for a period of time prior to transfusion to improve the RBC viability, e.g., for 6 to 24 hours prior to transfusion. For example, the current or field can be supplied to an transfusion container via electrodes and/or an external charging coil, which is activated during the transfusion, or in an embodiment before the transfusion for a duration effective to improve the external or surface polarity of the RBC, for example, 5 to 10 minutes. Where the RBC are treated for a sufficient duration prior to transfusion, the treatment can be continued at the same or a different, higher or lower current or field strength.

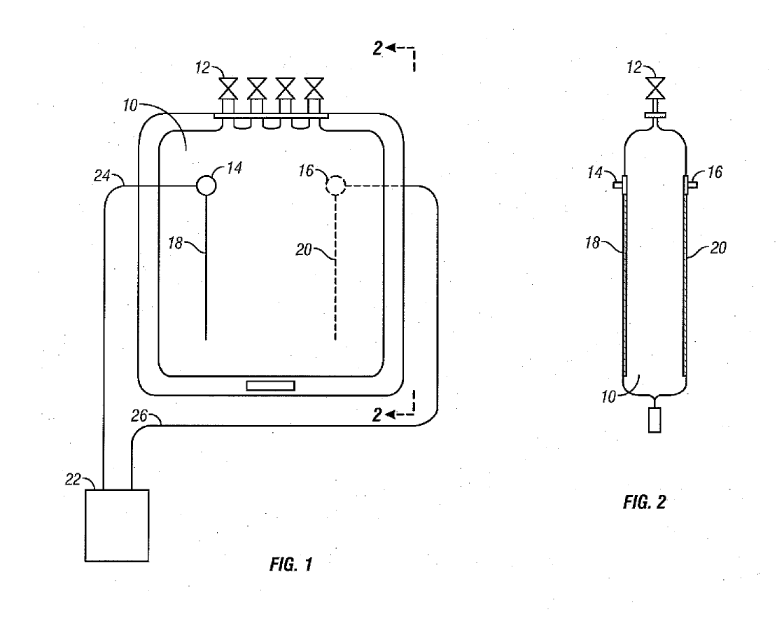

FIGS. 1 and 2 illustrate an embodiment of blood storage in a bag 10 or other storage medium container, for example, having one or more ports 12 for filling or removing fluid from the container 10. The container 10 is provided with a pair of electrodes 14, 16 that can include respective internal portions 18, 20 in electrical contact with the blood stored in the bag 10. For example, the internal portions 18, 20 can be flexible conductive wires adhered to or embedded at an inner surface of a wall of the blood bag 10. In another embodiment, the electrodes 14, 16 can be electrically connected via a biologically compatible wire mesh or wool within the bag 10 or other storage container. The internal electrodes 18, 20 can be positioned in an embodiment on opposite sides or ends of the container 10 to provide a relatively even current to the stored blood. An electrical current can be supplied from the controller 22 via conductors 24, 26 to the electrodes 14, 16 in electrical communication with the blood. In one embodiment, the electrical current, is effective to inhibit rouleaux aggregation and/or induce rouleaux disaggregation in the stored RBC.

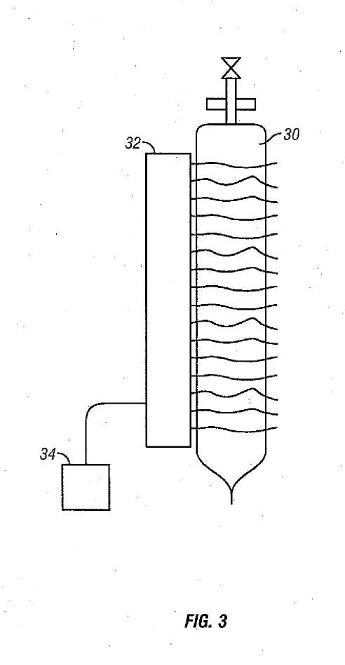

FIG. 3 illustrates electromagnetic field in accordance with an embodiment of the present invention being exposed to a storage medium container 30. Electromagnetic waves are pulsed through the bag 30 from an electromagnetic wave generator 32, as understood by those skilled in the art, adjacent to the bag 30. As used herein, the term “signal generator” means any device or circuit configured to produce an electric, magnetic or electromagnetic field; or an appropriate voltage or current (time varying or non-time varying) as understood by those skilled in the art. An example of a signal generator includes, without limitation, a power source, and associated circuitry capable of generating specific voltages and currents to one or more conductors; or capable of producing electric, magnetic or electromagnetic fields. Commercial embodiments of a signal generator include, but are not limited to the Hewlett-Packard 608 and the Fluke 6060 series of signal generators, a magnetron. Other commercial embodiments of a signal generator include, for example, the Geneva PF-211, PF-215 degaussers, Berner 3000 Mat, and the Bemer 300 Intensive Applicator. Embodiments of the signal generator can include, for example, operates over frequencies from High Frequency (3 to 30 MHz) to Very High Frequency (30-300 MHz) range as understood by those in the art.

A controller 34 can be used to set the desired frequency and amplitude of the electromagnetic waves. Embodiments of an magnetic field include, for example, of a range from about 0.5 to about 500 Gauss, or within a range of from about 10 to about 100 Gauss. In an embodiment, the magnetic field is pulsed at a frequency from about 0.001 to 10 Hz. Lower or higher intensity fields may also be used, for example, the magnetic field strength can range from greater than about 0.004, 1, 1.2, 10, 50, 100, 1000, 2500, 5000, or 10,000 Gauss or more.

The blood storage bags 10, 30 shown in FIGS. 1-3 can be stored in a temperature-controlled environment, e.g., a refrigerator or warming box. In one embodiment, the blood bags 10, 30 are stored in a refrigerator at 1 to 6° C. as is conventional. In another embodiment, the blood is maintained during storage at the normal body temperature of the animal from which it is taken, e.g., ±0.5° C., ±1° C., ±3° C., ±5° C., or ±10° C. In another embodiment, the blood storage bag or other container is maintained at human biological temperatures, e.g., from about 30° C. to about 42° C., or from about 30° C. to about 40° C., or from about 34° C. to about 40° C., or from about 35° C. to about 39° C., or about 37° C.±0.5° C. or ±1° C., or the like. Maintaining the temperature of the blood at approximately the biological temperature, such as for example at about a range of about 96.5 to about 99.5 degrees Fahrenheit, helps maintain the electromagnetic characteristics of the fluid. As understood by those skilled in the art, it is known that dielectric characteristics of water change dramatically with 10° C. or 20° C. temperature changes. Embodiments of the present invention advantageously cause white blood cells initially present in whole blood to stop or inhibit bacterial growth during the initial storage conditions at normal biological temperatures, and then the bag or container 10, 30 will maintain sterility by preventing contamination from outside the bag or container membrane, which is preferably biologically impermeable.

In one embodiment, the blood bags 10, 30 shovvn in FIGS. 1-3 can be stored on a rocker or vibrator to provide constant or periodic motion to inhibit settling and/or aggregation of the RBC.

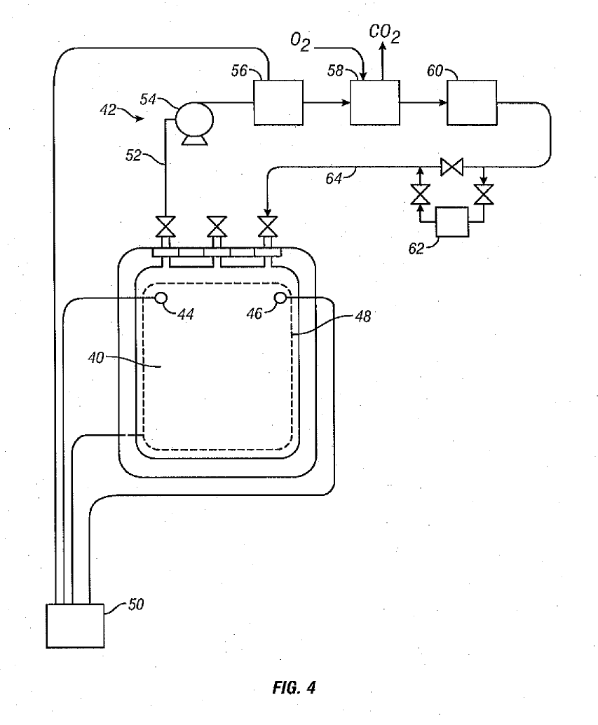

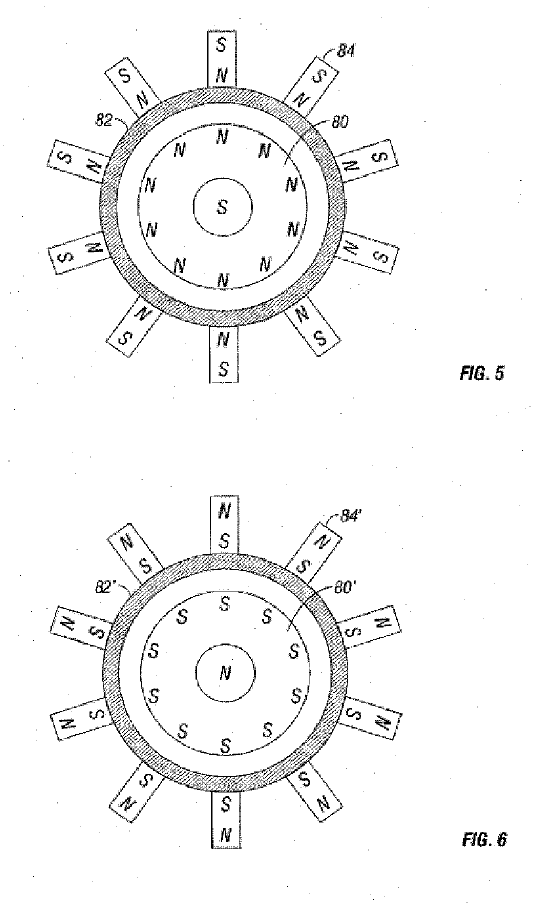

In another embodiment, as illustrated in FIG. 4, the storage device comprises at least one blood bag or container 40 and a flow circulation circuit 42. The blood bag 40 can be provided in one embodiment with electrodes 44, 46 and/or signal generator 48 operated by controller 50 for electromagnetic stimulation in the bag 40 as described above in reference to FIGS. 1-3. The blood bag 40 can additionally or alternatively be stored on a rocker for additional agitation and/or in a temperature controlled room or larger container.

A flow circulation circuit 42 comprises tubing 52 or other flow conduit and at least one pump 54 to continuously or periodically circulate the blood during storage. The pump 54, for example, can be magnetically shielded from the flow path to avoid or minimize exposure of the fluids to magnetic fields employed in the pump 54, especially static magnetic fields. Magnetically shielded pumps are disclosed in Applicant's applications U.S. Ser. No. 12/433,566, filed Apr. 30, 2009, U.S. 61/409,838 filed, Nov. 3, 2010, and U.S. 61/415,561 filed Nov. 19, 2010, which are hereby fully incorporated herein by reference. Embodiments of the pump 54 can also provide, for example, pressures similar to those in the cardiovascular system of the living being from which the blood is taken, e.g., 8 to 21.3 or 32 kPa (60 to 160 or 240 mm Hg) in the case of human blood, to simulate biological conditions and avoid damaging the RBC by excessive fluid pressure.

The flow circuit 42 may include, for example, one or more of an electromagnetic stimulation unit 56, respiration unit 58, dialysis unit 60, or any combination thereof. The electromagnetic stimulation unit 56, for example, can include electrodes to apply a current to blood flowing through the unit 56, an signal generator to apply a magnetic field to the blood flowing through the unit 56, or both. In an embodiment of the present invention, the electromagnetic stimulation unit 56 is integrated with the pump 54 to pump the blood in the flow path through the pump 54/unit 56, for example, wherein the magnetic field(s) in the stator and/or rotor of the pump 54 also function to provide the appropriate electromagnetic stimulation of the RBC.

In one embodiment, the electromagnetic stimulation unit 56 can provide small parallel flow channels with a diameter on the same order of magnitude as that of an RBC or capillary in the living being from which the blood was obtained, e.g. within 100 to 200%, preferably from 105 to 150% of the mean RBC diameter, or from 50 to 200% of the mean capillary diameter. For example, the simulated capillaries can have a cross sectional diameter of 5 to 20 microns or 8 to 15 microns. If desired, embodiments of the unit 56 can include appropriate supply and return manifolds to distribute the fluid flow through a plurality of the microehannels. The channels can be formed, for example, by placing grooves in a face of an inert plastic plate, sheet, film or block or other suitable material, and then securing the face to another face which can also be grooved. Where the grooves are semicircular in cross section and match with a similar groove in the opposite face, a circular channel will be formed; or where the opposite face is flat or planar the channel will be semicircular. Other shapes may be used, but circular cross sections matching the animal's capillary size and configuration are preferred. The number of channels should be sufficient to provide the total desired flow area, e.g., within 50 to 200% of the total cross sectional flow area in the tubing 52, 64. The length of the channels preferably are as short as possible to minimize pressure drop and hydraulic damage of the RBC as they “squeeze” through the capillary-mimicking channels, e.g., 0.5 to 5 cm.

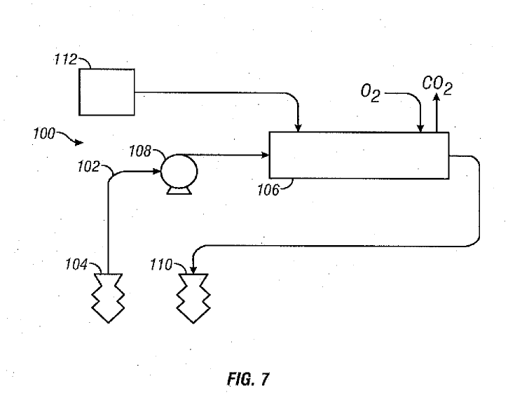

In one embodiment the electromagnetic stimulation unit 56 also includes at least one signal generator, which can be a degaussing (alternating) magnetic field, a static or step-pulsed deoxygenating field (magnetic north oriented toward the RBC or a cathodic electrical field), or an oxygenating field (magnetic south oriented toward the RBC or an anodic electrical field), or any combination thereof FIG. 5, for example, illustrates an RBC 80, which is fully oxygenated so that it has a north external polarity, passing through a capillary tube 82 wherein an external field 84 around the capillary 82 has a like polarity that tends to repel the RBC 80 so that sticking to the surface of the capillary 82 is less likely. In FIG. 6, the RBC 80′ is at least slightly deoxygenated so that it has a south external polarity, and the field 84′ has a south inward polarity. In one embodiment, especially at surfaces in contact with oxygenated blood, the magnets can have a “mild” magnetic field strength which is similar to that at the surface of normal erythrocytes. In this embodiment, the idea is to control the red blood cells from sticking together or to the exposed surfaces of the machine, but in one embodiment the field strength should not be so great as to induce oxygen release from the erythrocytes. A mild magnetic field can be attenuated in one embodiment by providing a relatively large flow cross section so that the magnets exert only an extremely minor field at the centerline or axis of the flow passage. In one embodiment, the magnets are provided at the oxygenator membranes, which may also optionally be heparinized as is known in the art.

In one embodiment, a first generator is provided to simulate electrobiological intracapillary deoxygenation in tissue and a second generator is provided downstream in series to simulate electrobiological intracapillary oxygenation in the lungs. In one embodiment, oxygen can be supplied and/or taken off via a gas permeable membrane in contact with the RBC in the microchannels just described, in the downstream respiration unit 58, or in the storage bag 40. Additionally or alternatively, if desired, carbon dioxide can be supplied and/or taken off via the same or different gas permeable membranes.

In another embodiment, an electrical current similar in voltage and current to that normally supplied at the atrioventricular node can be applied through the blood to and away from the oxygenator. In one embodiment the mild current is applied from an upstream electrode, to an electrode adjacent the oxygenator; in another embodiment from a downstream electrode, to the oxygenator electrode; and in another embodiment, the current is applied from both of the upstream and downstream electrodes to the common oxygenator electrode. In one embodiment, the current from the upstream and/or downstream electrodes is pulsed in a pattern similar to that of the atrioventricular node, and in another embodiment, the downstream electrode is pulsed approximately 0.02 seconds after the upstream electrode, corresponding to the current flow and pattern of the in vivo current from the atrioventricular node to the blood flowing between the heart and to/from the pulmonary capillaries.

In an embodiment, an electromagnetic, electric, magnetic field or combination thereof generated in the unit 56 is effective to inhibit rouleaux aggregation and/or induce rouleaux disaggregation in the RBC.

The respiration unit 58 can be or include, for example, a gas exchange unit to maintain desired levels of respiration gases, e.g., a membrane oxygenator to add and/or remove carbon dioxide and/or oxygen, to maintain oxygenation and carbon dioxide levels. Membrane oxygenators are well known for use in extracorporeal membrane oxygenation (ECMO) devices. In one embodiment the respiration unit 58 can be integrated with the electromagnetic stimulation unit 56, e.g., to provide electromagnetic stimulation during or in conjunction with gas exchange. For example, the electromagnetic stimulation (type, magnitude, frequency, polarity) associated with oxygen uptake and/or carbon dioxide release can mimic that which is biologically present in the air sac capillaries in the lungs, or the electromagnetic stimulation (type, magnitude, frequency, polarity) associated with oxygen release and/or carbon dioxide uptake can mimic that found in the tissues or organs other than the lungs. In one embodiment, the respiration unit 58 can include subunits in series to release oxygen/absorb carbon dioxide in a first subunit and to absorb oxygen/release carbon dioxide in the second subunit, as described above. In an embodiment, the RBC in return line 64 and blood bag 40 are more or less fully oxygenated, e.g. an oximetry of 98-100%.

Embodiments of the present invention can also include a dialysis unit 60 to remove waste components formed by the biological activity of the fluids, especially at normal biological temperatures. The fluid can also be supplemented with nutrients such as glucose or a slow release source of glucose can be added at the initial collection or processing of the blood or RBC in preparation for storage.

If desired, a side stream processing unit 62 can be provided in the return tubing 64. The unit 62 can include any type of hematological processing or testing equipment, or provide a sampling port for withdrawing specimens for testing or analysis. In one embodiment, the unit 62 includes an organ perfusion unit for maintaining the viability of the organ for transplant.

In an embodiment, the storage device can comprise two of the storage bags 40 to store oxygenated-state RBC and deoxygenated-state RBC, respectively. The blood under storage can be alternatingly pumped between the two bags in a first cycle to deoxygenate the RBC and in a second cycle to oxygenate the blood. The oxygenation and deoxygenation cycles can be provided in separate lines which are continuously operated in opposite directions more or less maintaining a constant blood volume in each storage container, or alternatively, the blood flow can be reversed in batch operations wherein the processing is alternated between batches between oxygenation and deoxygenation cycles. In one embodiment, the blood within the storage system can be pumped at a space velocity from 50% to 200% of the biological space velocity, e.g., from about 1 volume per 30 seconds to 1 volume per 2 minutes.

By repeatedly cycling the RBC through oxygenating and deoxygenating steps with similar biological hydrodynamic conditions, the electromagnetic health of the RBC can be viably maintained in storage up to about the same period of time as the RBC survives in vivo, or longer. To the extent the viability of RBC in vivo is a function of hydrodynamic conditions (where bumping and friction slowly degrade the RBC), the viability in storage can theoretically be further improved relative to biological conditions by providing comparatively improved hydrodynamic conditions, i.e., less bumping or friction by providing smooth walls, large radii turns, gradual diameter changes, elimination of obstructions and tortuous flow paths, maintaining laminar flow conditions, etc.

FIG. 7 relates to another embodiment wherein a heart lung machine or ECMO-type unit 100 is designed using an oxygenating flow circuit 102 that closely mimics pulmonary electromagnetic conditions in the body. Cardiopulmonary bypass devices remove deoxygenated blood from a venous cannula 104, through a series of tubes made from inert elastomeric materials and a membrane oxygenator 106, via a peristaltic or centrifugal pump 108, and returns the oxygenated blood to the patient at an arterial or venous cannula 110. The erythrocytes can be exposed to various electromagnetic, electric, and/or magnetic field in the prior art cardiopulmonary bypass process, e.g. from the pump or adjacent wires or other conductors where an electrical current is present, and in some cases these electromagnetic, electric, and/or magnetic field can induce the wrong polarity (“contramagnetic”) or insufficient (“hypomagnetic”) or excessive (“hypermagnetic”) strength (collectively, “dysmagnetic”) relative to erythrocytes passing normally (“eumagnetic”) through the right atrium and ventricle, to the pulmonary capillaries and then to the left atrium and ventricle, so that the blood is not properly magnetized and/or not properly oxygenated. It is believed that the dysmagentic conditions in cardiopulmonary bypass can contribute to Rouleaux formation, “sticky” blood that accumulates on the tubing, membrane and pump impeller surfaces, and may also cause or contribute to postperfusion syndrome, hemolysis, capillary leak syndrome, blood clotting in the oxygenator, air embolism, microembolic events, and the like.

In one embodiment, the tubing in the flow circuit 102 of FIG. 7 is constructed as shown in FIG. 5 and/or FIG. 6 discussed above, i.e., with an array of magnets or magnetic field generator 84, 84′ at the tubing wall 82, 82′, or spaced from but sufficiently close to the wall 82, 84 to induce a magnetic field into the tubing. In the case of the tubing 82, 82′, which has a circular section, the array is radial; however, in the case of a flat surface such as at an oxygenation membrane, the array may be planar. The magnetic field 84, 84′ can be static or electromagnets and are aligned with ends of like polarity facing toward the fluid flowing through the flow passage 102 and transporting the erythrocytes 80, 80′.

The magnets 84, 84′ in one embodiment exert a magnetic field onto the red blood cells 80, 80′, preferably of a like polarity with respect to the surface polarity of the erythrocytes 80, 80′. In one embodiment, the dipole orientation of the magnets 84, 84′ is the same as that of the surface of the red blood cell 80, 80′ so that there is a repulsion of the red blood cell 80, 80′ away from the inner surface of the tubing wall 82, 82′. For example, in FIG. 5 where the erythrocytes 80 have a north external polarity, the magnets 84 can be oriented with the north poles facing into the wall 82 of the flow passage 102 to push the erythrocytes away from the wall 82 and thereby inhibit adhesion or clotting at the surface, thus facilitating prevention of the accumulation of any “sticky” blood cells. In an alternative embodiment, an oscillating magnetic field is applied to alternatingly attract and repel the RBC to inhibit adhesion.

In one embodiment, especially at surfaces in contact with oxygenated blood, the magnets 84 can have a “mild” magnetic field strength which is similar to that at the surface of normal erythrocytes. In this embodiment, the idea is to control the red blood cells 80 from sticking together or to the exposed surfaces of the machine, but the field strength should not be so great as to induce oxygen release from the erythrocytes. A mild magnetic field, as understood by those skilled in the art, can be attenuated in one embodiment by providing a relatively large flow cross section, e.g., 1-25 mm inside diameter, so that the magnets 80 exert only an extremely minor field at the centerline or axis of the flow passage 102. In one embodiment, the magnets 84 are provided at the oxygenator membranes in unit 106, which may also optionally be heparinized as is known in the art.

In another embodiment with reference to FIG. 7, the oxygenator 106 can include an electromagnetic stimulator similar in design and function to the unit 56 discussed above in connection with FIG. 4, and controller 110 to provide the electromagnetic stimulation as desired, e.g., application of a magnetic field or electric current, preferably in coordination with the oxygenation function of the oxygenator 106. In one embodiment, an electrical current similar in voltage and current to that normally supplied at the atrioventricular node can be applied through the blood passing through the tubing 102 leading to and away from the oxygenator 106. For example, the electrodes can be electrically connected via a biologically compatible wire mesh or wool within the tubing 102 or oxygenator 106. In one embodiment the mild current is applied from an upstream electrode, e.g. adjacent the venous cannula 104, to an electrode adjacent the oxygenator; in another embodiment from a downstream electrode, e.g., adjacent the arterial cannula 110, to the oxygenator electrode; and in another embodiment, the current is applied from both of the upstream and downstream electrodes to the common oxygenator electrode. In one embodiment, the current from the upstream and/or downstream electrodes is pulsed in a pattern similar to that of the atrioventricular node, and in another embodiment, the downstream electrode is pulsed approximately 0.02 seconds after the upstream electrode, corresponding to the current flow and pattern of the in vivo current from the atrioventricular node to the blood flowing between the heart and to/from the pulmonary capillaries.

Many modifications and other embodiments of the invention will come to the mind of one skilled in the art having the benefit of the teachings presented in the foregoing descriptions and the associated drawings. Therefore, it is to be understood that the invention is not to be limited to the illustrated embodiments disclosed, and that modifications and other embodiments are intended to be included within the scope of the appended claims.

What is claimed:

1. A method for storing fluids to thereby enhance the shelf life and viability of the fluids, the method comprising:
providing a storage medium containing fluids, the storage medium storing the fluid outside a living being;
providing a signal generator that operates over frequencies from High Frequency to Very High Frequency range;
exposing the storage medium containing the fluids to an electric, magnetic, or electromagnetic field provided by the signal generator for an exposure time to thereby slow degeneration of one or more components of the fluids during storage of the fluids outside the living being.

2. A method as defined in claim 1, wherein the storage medium is exposed to a combination of electric, magnetic, or electromagnetic fields.

3. A method as defined in claim 1 and claim 2, wherein storage medium is exposed to the magnetic field in a range of about 0.5 to 100 Gauss, the electric field in a range of about 3 to 300 volts/m, or the electromagnetic field in a range of about 15 to 100 watts/m².

4. A method as defined in claim 1, the method further comprising:
agitating the fluids to provide constant or periodic motion to reduce the risk of settling and aggregation of one or more cells of the fluids and to mitigate problems associated with formation of hot spots.

5. A method as defined in claim 1, the method further comprising:
circulating the fluids continuously to provide constant motion to reduce the risk of settling and aggregation of one or more cells of the fluids and also to mitigate problems associated with formation of hot spots.

6. A method as defined in claim 5, wherein a pump circulates the fluids at a rate of less than about 150 milliliters per minute to mitigate damaging the one or more cells of the fluids.

7. A method as defined in claim 5 and claim 6, wherein a polarity of the electric, magnetic or electromagnetic field has a like polarity of one or more cells of the fluids to thereby cause the one or more cells be repelled from the field to thereby reduce a risk of the one or more cells sticking to a surface of the storage medium, adhesions, or clotting.

8. A method as defined in claim 1 and claim 7, the method further comprising:
maintaining temperature of the fluids at a range of about 96.5 to about 99.5 degrees Fahrenheit.

9. A method as defined in claim 1, wherein exposing the storage medium containing the fluids to the electric, magnetic or electromagnetic field includes pulsing the field at predetermined intervals.

10. A method as defined in claim 1, wherein the exposure time is at least 35 days, and wherein the storage medium containing the fluids is exposed to the electric, magnetic or electromagnetic field continuously or at preselected intervals for at least 35 days.

11. A method as defined in claim 1 and claim 7, wherein the exposure time is at least 42 days, and wherein the storage medium containing the fluids is exposed to the electric, magnetic or electromagnetic field continuously or at preselected intervals for at least 42 days.

12. A method as defined in claim 1, the method further comprising:
circulating the fluids to provide constant or periodic motion to reduce the risk of settling and aggregation of the fluids and mitigate problems associated with hot spots;
maintaining temperature of the fluids at about a range of about 96.5 degrees Fahrenheit; and
wherein the storage medium is exposed to a combination of electric, magnetic, or electromagnetic fields.

13. A system for storing fluids, the system comprising:
a storage medium container containing whole blood or any components thereof to define fluids, the storage medium container storing the fluid outside a living being;
a pump for circulating the fluid through the storage medium container and one or more tubes, the one or more tubes being in fluid communication with the fluid; and
an signal generator selectively producing an electric, magnetic, or electromagnetic field to thereby expose the fluid or portions thereof to the electric, magnetic or electromagnetic field for an exposure time to thereby slow degeneration of one or more components of the fluids during storage of the fluids outside the living being.

14. A system as defined in claim 13, wherein the pump circulates the fluids at a rate of less than about 150 milliliters per minute to mitigate damaging one or more cells of the fluids, and wherein the storage medium is exposed to a combination of electric, magnetic, or electromagnetic fields.

15. A system as defined in claims 13 and 14, wherein a polarity of the electric, magnetic or electromagnetic field, or combination thereof, has a like polarity of one or more cells of the fluids to thereby cause the one or more cells to be repelled from the field and to reduce a risk of the one or more cells sticking to a surface of the storage medium, adhesions, or clotting; and maintaining temperature of the fluids at about a range of about 96.5 degrees Fahrenheit.
